(19) United States
(12) Patent Application Publication
(10) Pub. No.: US 2024/0415402 A1
(43) Pub. Date: Dec. 19, 2024

(54) MEASURING APPARATUS AND MEASURING METHOD

(71) Applicant: TAIYO YUDEN CO., LTD., Tokyo (JP)

(72) Inventor: Takayuki SEKIGUCHI, Gunma (JP)

(73) Assignee: TAIYO YUDEN CO., LTD., Tokyo (JP)

(21) Appl. No.: 18/787,793

(22) Filed: Jul. 29, 2024

Related U.S. Application Data
(63) Continuation of application No. PCT/JP2022/043829, filed on Nov. 28, 2022.

(30) Foreign Application Priority Data
Jan. 31, 2022 (JP) 2022-013622

(51) Int. Cl. A61B 5/0537; A61B 5/145

(52) U.S. Cl. CPC A61B 5/0537; A61B 5/14532; A61B 5/1451

(57) ABSTRACT

A measuring apparatus includes a first substrate, an oscillator circuit, and an arithmetic circuit. The first substrate is made of a dielectric and has a first signal line and a ground conductor. A living body is to be pressed against the first signal line. The oscillator circuit produces a first signal of alternating current. The arithmetic circuit acquires biological information based on a comparison between a second signal and a third signal. The second signal corresponds to the first signal that passes through the first signal line. The third signal corresponds to the first signal that does not pass through the first signal line.

### CROSS-REFERENCE TO RELATED APPLICATIONS

This application is a continuation of PCT International Application No. PCT/JP2022/043829 filed on Nov. 28, 2022, which designates the United States, incorporated herein by reference, and which claims the benefit of priority from Japanese Patent Application No. 2022-013622, filed on Jan. 31, 2022, incorporated herein by reference.

### FIELD

Embodiments described herein relate to a measuring apparatus and a measuring method.

### BACKGROUND

Some biological information, such as blood sugar levels, is measured using blood sampling or other invasive means. In recent years, however, there has been a growing demand for technology that can measure biological information as non-invasively as possible, in order to reduce physical burden on subjects and reduce the risk of contracting infectious diseases. The related technologies are described, for example, in: Japanese Patent No. 5600759; Publication of Japanese Translation of PCT Application No. 2009-500096; and Publication of Japanese Translation of PCT Application No. 2021-502880.

There are needs for a measuring apparatus and a measurement method that can measure biological information non-invasively.

### SUMMARY

According to an aspect of the present invention, a measuring apparatus includes a first substrate, an oscillator circuit, and an arithmetic circuit. The first substrate is made of a dielectric and has a first signal line and a ground conductor. A living body is to be pressed against the first signal line. The oscillator circuit produces a first signal of alternating current. The arithmetic circuit acquires biological information based on a comparison between a second signal and a third signal. The second signal corresponds to the first signal that passes through the first signal line. The third signal corresponds to the first signal that does not pass through the first signal line.

### BRIEF DESCRIPTION OF THE DRAWINGS

FIG. 1 is a diagram illustrating the permittivity of a plurality of aqueous solutions with different glucose concentrations;

FIG. 2 is a schematic diagram illustrating an example of the configuration of a blood sugar level measuring apparatus of a first embodiment;

FIG. 3 is a perspective view of a sensor in the first embodiment;

FIG. 4 is a cross-sectional view of the sensor in the first embodiment cut in the YZ plane;

FIG. 5 is a schematic diagram illustrating an electromagnetic field distribution when the subject's skin is pressed against a first signal line in the first embodiment;

FIGS. 6A and 6B are schematic diagrams illustrating change in wavelength of an AC signal passing through the first signal line when the subject's skin is pressed against the first signal line of the sensor in the first embodiment during fasting and after eating;

FIG. 7 is a schematic diagram illustrating an example of temporal transition of the change in wavelength of the AC signal flowing through the first signal line when a subject touches the first signal line of the sensor in the first embodiment;

FIG. 8 is a flowchart illustrating an example of the operation of the blood sugar level measuring apparatus of the first embodiment;

FIG. 9 is a diagram illustrating an example of the relationship between change in phase and change in frequency of a sensor pass signal in the first and second embodiments;

FIG. 10 is a schematic diagram illustrating an example of the configuration of a blood sugar level measuring apparatus of the second embodiment;

FIG. 11 is a cross-sectional view of the sensor of a first modification cut in the YZ plane;

FIGS. 12A to 12C are cross-sectional views of the sensor of a second modification cut in the YZ plane;

FIGS. 13A to 13C are schematic diagrams illustrating the shape of the first signal line of a third modification;

FIG. 14 is a diagram of a sensor unit of a fourth modification as viewed from the positive side in the Z direction;

FIG. 15 is a diagram of the sensor unit of the fourth modification as viewed from the negative side in the Z direction;

FIG. 16 is a cross-sectional view of the sensor unit of the fourth modification cut in the XZ plane; and

FIG. 17 is a diagram illustrating transmission paths of a sensor pass signal and a local signal when the fourth modification is applied to the first embodiment.

### DETAILED DESCRIPTION

The concentration of glucose in the interstitial fluid of the dermal layer is known to correlate with the concentration of glucose in the blood, that is, blood sugar level.

Furthermore, the permittivity of liquid varies depending on the concentration of glucose in the liquid.

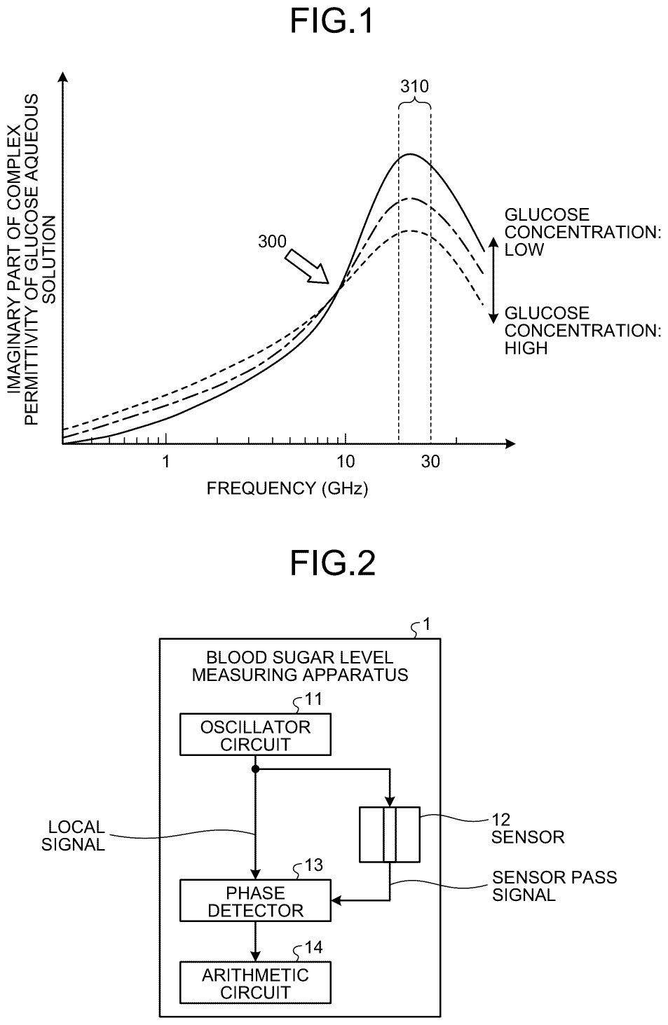

FIG. 1 is a diagram illustrating the permittivity of a plurality of aqueous solutions with different glucose concentrations. In FIG. 1, the vertical axis represents the imaginary part of complex permittivity and the vertical axis represents the frequency.

As illustrated in FIG. 1, the imaginary part of complex permittivity has different frequency characteristics depending on glucose concentrations. On the high frequency side of an inflection point 300 near 10 GHz, the imaginary part of complex permittivity is smaller as the glucose concentration in the aqueous solution is higher. In a certain frequency range 310, the dependence of the imaginary part of complex permittivity on the glucose concentration is significantly large.

It is noted that the real part of complex permittivity varies with a tendency opposite to that of the imaginary part of complex permittivity. In other words, on the high frequency side of the inflection point 300, the real part of complex permittivity is larger as the glucose concentration in the aqueous solution is higher. The dependence of the real part of complex permittivity on the glucose concentration is large in the certain frequency range 310, which is the same as in the imaginary part of the complex permittivity. Hereafter, the permittivity refers to the real part of complex permittivity.

The permittivity of human skin, specifically the dermal layer, has a dependence similar to the glucose concentration dependence illustrated in FIG. 1 on the glucose concentration in the interstitial fluid of the dermal layer. As noted above, there is a correlation between glucose concentrations in the interstitial fluid of the dermal layer and blood sugar levels. Thus, if a value related to the permittivity of the skin can be obtained, the blood sugar level can be estimated. The measuring apparatus of embodiments estimates blood sugar levels based on a value related to the permittivity of the skin.

In embodiments, a sensor having a structure of a transmission line with a signal line on a substrate is used as a sensor for obtaining a value related to the permittivity of the skin. An AC signal is fed to the signal line, and when a subject touches the signal line, the wavelength of the AC signal flowing through the signal line changes in accordance with the permittivity of the skin that touches the signal line. This change in wavelength is related to the permittivity of the skin. The measuring apparatus of embodiments acquires a measurement value of blood sugar level based on the change in wavelength of the AC signal flowing through the signal line. Since blood sugar levels can be measured simply by the subject touching the signal line, non-invasive blood sugar level measurement can be implemented.

The measuring apparatus of embodiments can be implemented in any device. The measuring apparatus of embodiments can measure blood sugar levels non-invasively and therefore can be implemented, for example, in a wearable device such as a smartwatch. The measuring apparatus of embodiments may be configured as a stationary measuring apparatus.

The biological information to be measured by the measuring apparatus of embodiments is not limited to blood sugar levels. Variations of the measurement target will be discussed later.

Referring to the attached drawings, a blood sugar level measuring apparatus which is an example of the measuring apparatus according to embodiments and a method of measuring blood sugar levels will be described below. The present invention is not intended to be limited by these embodiments.

### First Embodiment

FIG. 2 is a schematic diagram illustrating an example of the configuration of a blood sugar level measuring apparatus 1 of a first embodiment. As illustrated in this figure, the blood sugar level measuring apparatus 1 includes an oscillator circuit 11, a sensor 12, a phase detector 13, and an arithmetic circuit 14.

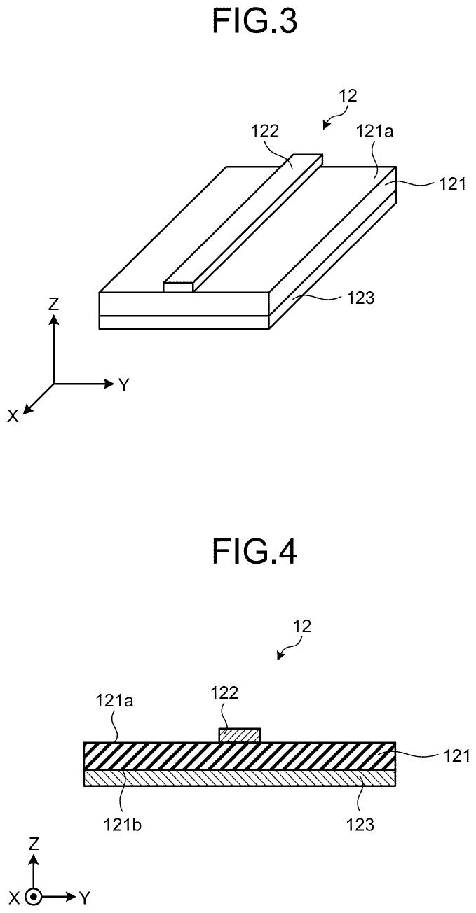

In the first embodiment, the sensor 12 has a structure similar to a microstrip line which is a kind of transmission line. Referring to FIG. 3 and FIG. 4, an example configuration of the sensor 12 in the first embodiment will be described.

FIG. 3 is a perspective view of the sensor 12 in the first embodiment. FIG. 4 is a cross-sectional view of the sensor 12 in the first embodiment cut in the YZ plane. The sensor 12 has the shape of a rectangular flat plate. In FIG. 2, FIG. 3, and some subsequent figures, the positional relationship and orientation of the components of the sensor 12 are illustrated, where the thickness direction of the sensor 12 is the Z direction, the direction in which one side of the rectangular shape of the sensor 12 extends is the X direction, and the direction orthogonal to the X direction and in which another side of the rectangular shape of the sensor 12 extends is the Y direction. The shape of the sensor 12 is not necessarily rectangular.

The sensor 12 includes a first substrate 121 made of a dielectric. The material of the first substrate 121 can be made of, for example, a common substrate material such as polytetrafluoroethylene (PTFE) or polyimide.

On a part of one face 121a of the first substrate 121, a first signal line 122 made of a conductor and extending in the X direction with a certain thickness and width is provided to pass through approximately the center when the first substrate 121 is viewed in a plan view. On the other face 121b of the first substrate 121, a ground conductor 123 is provided and formed over the entire surface of the face 121b. It is not necessarily formed on the entire surface. The first signal line 122 and the ground conductor 123 are made of, for example, a material with high electrical conductivity, such as copper or gold.

The face 121a of the first substrate 121 is an example of the first face. The face 121b of the first substrate 121 is an example of the second face.

A measurement target, that is, in this case, the subject's skin, is pressed against the first signal line 122 from the positive side in the Z direction, with an AC electrical signal being fed.

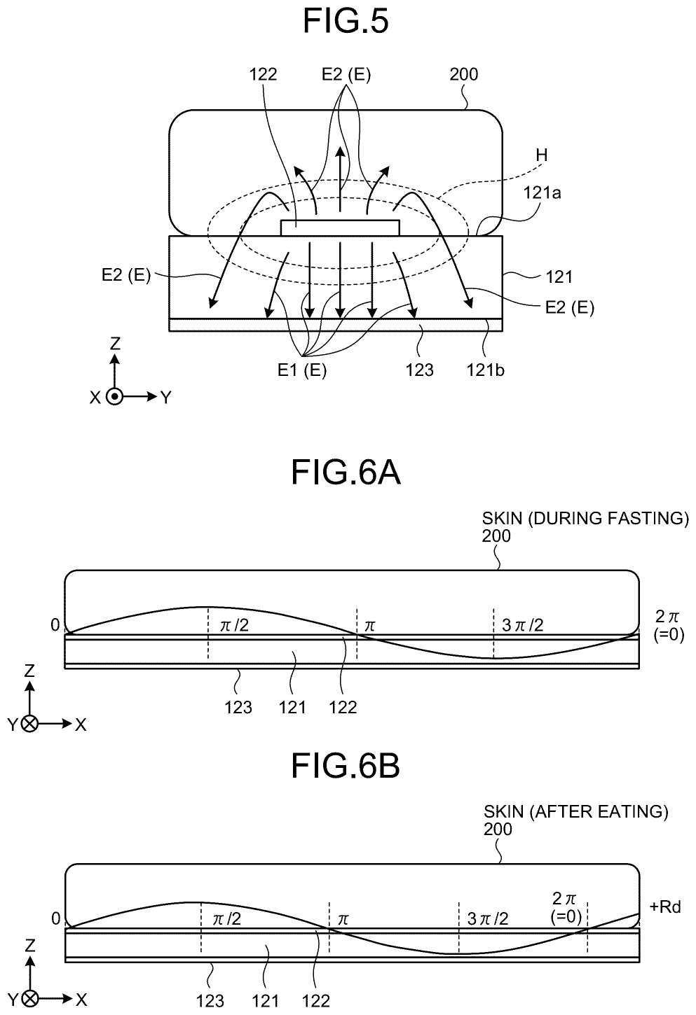

FIG. 5 is a schematic diagram illustrating an electromagnetic field distribution when the subject's skin 200 is pressed against the first signal line 122 in the first embodiment. The solid arrow E indicates an electric field vector, and the dotted line H indicates a magnetic field distribution.

When an AC signal is flowing through the first signal line 122, electric field vectors E are formed. Most of electric field vectors E1 are concentrated between the first signal line 122 and the ground conductor 123, but there are some electric field vectors E2 that have a path from the face 121a to the outside of the first substrate 121. When the skin 200 comes into contact with the first signal line 122, the electric field vectors E2 pass through the skin 200 to change the wavelength of the AC signal flowing through the first signal line 122. The wavelength shortening ratio k is generally inversely proportional to the square root of the relative permittivity and is expressed by the following equation (1).

$$k = 1 / \sqrt{{ε}_{r}} \qquad ( 1 )$$

Depending on the relative permittivity of the object through which the electric field vector E2 passes, the wavelength of the AC signal when the object is in contact with the first signal line 122 changes from the wavelength of the AC signal when nothing touches the first signal line 122.

When subjects eat, blood sugar levels increase and glucose concentrations in the interstitial fluid of the dermal layer increase. Then, in a specific frequency range (for example, the range on the high frequency side of the inflection point 300 in FIG. 1), the higher the glucose concentration, the higher the permittivity. Thus, as the subject's blood sugar level increases, the shortening ratio k becomes smaller and the wavelength of the AC signal passing through the first signal line 122 becomes shorter.

FIGS. 6A and 6B are schematic diagrams illustrating change in wavelength of an AC signal passing through the first signal line 122 when the subject's skin is pressed against the first signal line 122 of the sensor 12 in the first embodiment during fasting and after eating.

For ease of understanding, it is assumed that the wavelength of the AC signal at the permittivity of the skin 200 when the subject is fasting is equal to the length (here, the length in the X direction) from the input end to the output end of the first signal line 122. In other words, when the subject touches the first signal line 122 during fasting, the AC signal is transmitted at a wavelength equal to the length of the first signal line 122, as illustrated in FIG. 6A. Thus, when the phase of the AC signal at the input end of the first signal line 122 is 0 radians, the phase of the AC signal at the output end of the first signal line 122 is 0 radians.

When the subject eats and touches the first signal line 122 with the blood sugar level increased, the wavelength is shortened, as illustrated in FIG. 6B. Thus, when the phase of the AC signal input to one end of the first signal line 122 is 0 radians, the phase of the AC signal output from the other end of the first signal line 122 is advanced by the amount of the shortened wavelength, compared to the phase illustrated in FIG. 6A. The amount of advance relative to the phase in the fasting state is denoted as the amount of phase advance Rd.

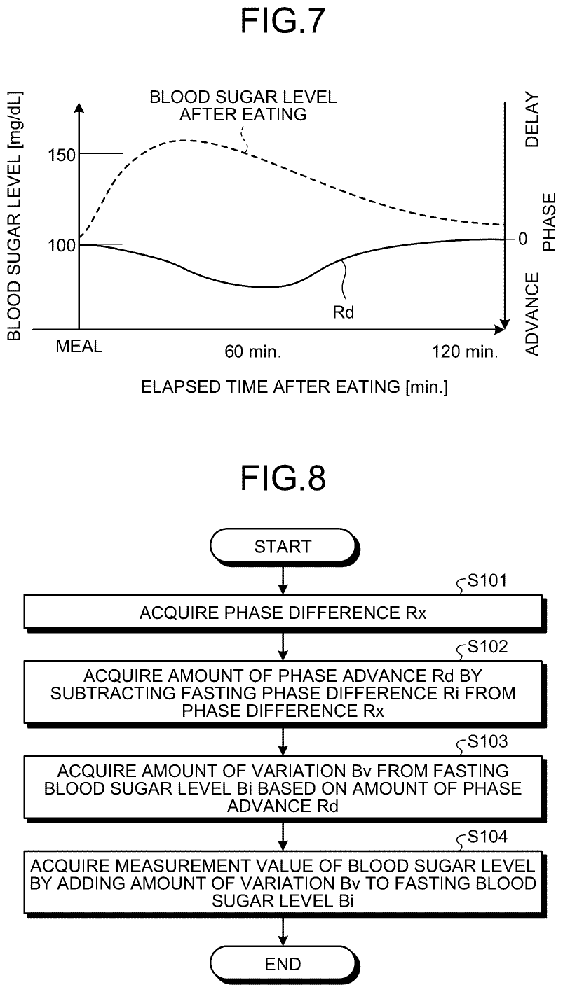

FIG. 7 is a schematic diagram illustrating an example of temporal transition of the change in wavelength of the AC signal flowing through the first signal line 122 when a subject touches the first signal line 122 of the sensor 12 in the first embodiment. In this figure, the horizontal axis indicates the elapsed time after eating. The left vertical axis indicates the blood sugar level, and the right vertical axis indicates the phase.

As illustrated in FIG. 7, as the blood sugar level increases after eating, the amount of phase advance Rd increases in accordance with the increase in blood sugar level. Then, as the blood sugar level begins to fall, the amount of phase advance Rd decreases. In this way, the amount of phase advance Rd changes in conjunction with the blood sugar levels.

The blood sugar level measuring apparatus 1 calculates the amount of phase advance Rd and calculates the measurement value of blood sugar level based on the amount of phase advance Rd.

The description returns to FIG. 2.

The oscillator circuit 11 produces an AC signal with a single frequency. The frequency of the AC signal produced by the oscillator circuit 11 is a frequency selected from the range in which the permittivity of the skin can change in accordance with blood sugar levels. The oscillator circuit 11 produces an AC signal with a frequency selected from the range 310 in FIG. 1, for example. The frequency of the AC signal produced by the oscillator circuit 11 may be selected from a range other than the range 310.

The AC signal transmission line connected to the oscillator circuit 11 is split into two. One of the two split transmission lines is connected to the input end of the first signal line 122, and the other of the two split transmission lines is connected to the phase detector 13. The output end of the first signal line 122 is then connected to the phase detector 13. Thus, the AC signal that passes through the first signal line 122 and the AC signal that does not pass through the first signal line 122 are input to the phase detector 13. The AC signal that passes through the first signal line 122 and is input to the phase detector 13 is denoted as a sensor pass signal. The AC signal that does not pass through the first signal line 122 and is input to the phase detector 13 is denoted as a local signal.

The AC signal produced by the oscillator circuit 11 is an example of the first signal. The AC signal that passes through the first signal line 122, that is, the sensor pass signal, is an example of the second signal. The AC signal that does not pass through the first signal line 122, that is, the local signal, is an example of the third signal.

The phase detector 13 detects the phase difference Rx between the sensor pass signal and the local signal and inputs the detected value of the phase difference to the arithmetic circuit 14. The phase detector 13 may be referred to as a phase comparator.

The arithmetic circuit 14 is a processor that performs predetermined arithmetic operations. The arithmetic circuit 14 is, for example, a microcomputer unit including a central processing unit (CPU) and a memory that stores a computer program. The CPU executes arithmetic operations based on the computer program. The arithmetic circuit 14 may be configured with a hardware circuit such as a field-programmable gate array (FPGA) or an application specific integrated circuit (ASIC).

The arithmetic circuit 14 acquires the measurement value of blood sugar level of the subject based on the phase difference Rx input from the phase detector 13.

The arithmetic circuit 14 can output the measurement value of blood sugar level by any method. When the blood sugar level measuring apparatus 1 includes an output device such as a display device or a speaker, the arithmetic circuit 14 may output the measurement value of blood sugar level to the output device such as a display device or a speaker. When the blood sugar level measuring apparatus 1 includes a memory, the measurement value of blood sugar level may be output to the memory. When the blood sugar level measuring apparatus 1 includes a communication device, the arithmetic circuit 14 may output the measurement value of blood sugar level to an external device via the communication device.

FIG. 8 is a flowchart illustrating an example of the operation of the blood sugar level measuring apparatus 1 of the first embodiment. The sequence of operation illustrated in this figure is performed while the subject is touching the first signal line 122 to measure blood sugar levels.

The phase detector 13 acquires the phase difference Rx between the sensor pass signal and the local signal (S101). The phase difference Rx is input to the arithmetic circuit 14.

The arithmetic circuit 14 acquires the amount of phase advance Rd by subtracting the fasting phase difference Ri, which is the phase difference Rx between the sensor pass signal and the local signal when the subject is in the fasting state, from the phase difference Rx acquired at S101 (S102).

It is assumed that the fasting phase difference Ri is measured in advance and stored in the arithmetic circuit 14 or in a memory accessible by the arithmetic circuit 14. For example, when the blood sugar level measuring apparatus 1 is implemented in a wearable device, the arithmetic circuit 14 stores the transition of the phase difference Rx in a period during which the subject wears the blood sugar level measuring apparatus 1 all day. The arithmetic circuit 14 then stores the lowest value of the phase difference Rx as the fasting phase difference Ri. The method of acquiring the fasting phase difference Ri is not limited to this.

It is also assumed that the fasting blood sugar level Bi, which is the blood sugar level when the subject is in the fasting state, is measured in advance, in the same manner as the fasting phase difference Ri, and stored in the arithmetic circuit 14 or in a memory accessible by the arithmetic circuit 14, in association with the fasting phase difference Ri. The method of measuring the fasting blood sugar level Bi is not limited to a specific method. The fasting blood sugar level Bi can be measured, for example, by blood sampling.

Following S102, the arithmetic circuit 14 acquires the amount of variation Bv of blood sugar level from the fasting blood sugar level Bi, based on the amount of phase advance Rd (S103).

For example, a calibration curve (denoted as the first calibration curve) expressing the relationship between the amount of phase advance Rd and the amount of variation Bv is acquired in advance by simulation or experiments using one or more subjects. The first calibration curve may be a function or may be information in a table form. The first calibration curve is stored in advance in the arithmetic circuit 14 or in a memory accessible by the arithmetic circuit 14. At S103, the arithmetic circuit 14 acquires the amount of variation Bv at the time of execution of S103, based on the amount of phase advance Rd acquired at S102 and the first calibration curve.

Following S103, the arithmetic circuit 14 acquires the measurement value of blood sugar level by adding the amount of variation Bv acquired at S103 to the fasting blood sugar level Bi (S104). The operation of the blood sugar level measuring apparatus 1 then ends.

The operation for acquiring a measurement value of blood sugar level illustrated in FIG. 8 has been described only by way of example. The operation for acquiring a measurement value of blood sugar level can be modified in various ways.

For example, a calibration curve (denoted as the second calibration curve) expressing the relationship between the phase difference Rx and blood sugar levels is acquired in advance by simulation or experiments using one or more subjects and stored in advance in the arithmetic circuit 14 or in a memory accessible by the arithmetic circuit 14. The arithmetic circuit 14 may then acquire the measurement value of blood sugar level, based on the phase difference Rx acquired at S101 and the second calibration curve.

Alternatively, the arithmetic circuit 14 may calculate the permittivity εₓ of the skin based on the phase difference Rx and acquire the measurement value of blood sugar level based on the permittivity εₓ of the skin. For example, the arithmetic circuit 14 converts the phase difference Rx into the permittivity εₓ of the skin, for example, based on the following equation (2). Here, a and b are coefficients obtained based on the relationship between the permittivity and the phase difference Rx, which is acquired in advance by pressing a sample with a known permittivity against the first signal line 122 to acquire the phase difference Rx.

$${ε}_{x} = a \times Rx + b \qquad ( 2 )$$

The arithmetic circuit 14 then acquires the measurement value of blood sugar level based on the permittivity εₓ of the skin. For example, a calibration curve (denoted as the third calibration curve) expressing the relationship between the permittivity εₓ of the skin and blood sugar levels is acquired in advance by simulation or experiments using one or more subjects and stored in advance in the arithmetic circuit 14 or in a memory accessible by the arithmetic circuit 14. The arithmetic circuit 14 acquires the measurement value of blood sugar level based on the permittivity εₓ of the skin acquired by equation (2) and the third calibration curve.

In the operation illustrated in FIG. 8, the arithmetic circuit 14 calculates the measurement value of blood sugar level relative to the subject's fasting blood sugar level Bi, because the wavelength of the AC signal transmitted through the first signal line 122 can vary with subject's race, gender, individual differences in body composition, and the like, even when the blood sugar level is the same. Since the fasting phase difference Ri and the fasting blood sugar level Bi of the subject are acquired in advance and the measurement value of blood sugar level is calculated relative to these values, accurate blood sugar level measurement is possible even when the subject's race, gender, individual differences in body composition, and the like vary.

Another example of the operation that takes into account the subject's race, gender, individual differences in body composition, and the like is the operation described below. For example, a glucose tolerance test is performed on a subject, and during the glucose tolerance test, a calibration curve (denoted as the fourth calibration curve) is created that represents the relationship between the phase difference Rx and the blood sugar level obtained by blood sampling or any other blood sugar level measuring apparatus. The fourth calibration curve is stored in the arithmetic circuit 14 or in a memory accessible by the arithmetic circuit 14. When the phase difference Rx obtained at S101 is input, the arithmetic circuit 14 acquires the measurement value of blood sugar level using the phase difference Rx and the fourth calibration curve. According to this example of operation, since the fourth calibration curve created for each subject is used, accurate blood sugar level measurement is possible even when the subject's race, gender, individual differences in body composition, and the like vary.

Even when the subject's race, gender, individual differences in body composition, and the like are taken into account, the arithmetic circuit 14 may calculate the permittivity εₓ of the skin based on the phase difference Rx and acquire the measurement value of blood sugar level based on the permittivity εₓ of the skin.

In this way, according to the first embodiment, the blood sugar level measuring apparatus 1 includes the sensor 12 including the first substrate 121 made of a dielectric and having the ground conductor 123 and the first signal line 122 against which a living body is to be pressed, the oscillator circuit 11 that produces an AC signal, the phase detector that detects a phase difference between a sensor pass signal which is an AC signal passing through the first signal line 122 and a local signal which is an AC signal not passing through the first signal line 122, and the arithmetic circuit 14 that acquires the measurement value of blood sugar level based on the phase difference.

Thus, blood sugar levels can be measured non-invasively.

According to the first embodiment, the sensor 12 has a structure in which the first signal line 122 is provided on the face 121a of the first substrate 121 and the ground conductor 123 is provided on the face 121b opposite the face 121a. Examples of the structure of the sensor 12 are not limited to this. Modifications of the sensor 12 will be described later.

### Second Embodiment

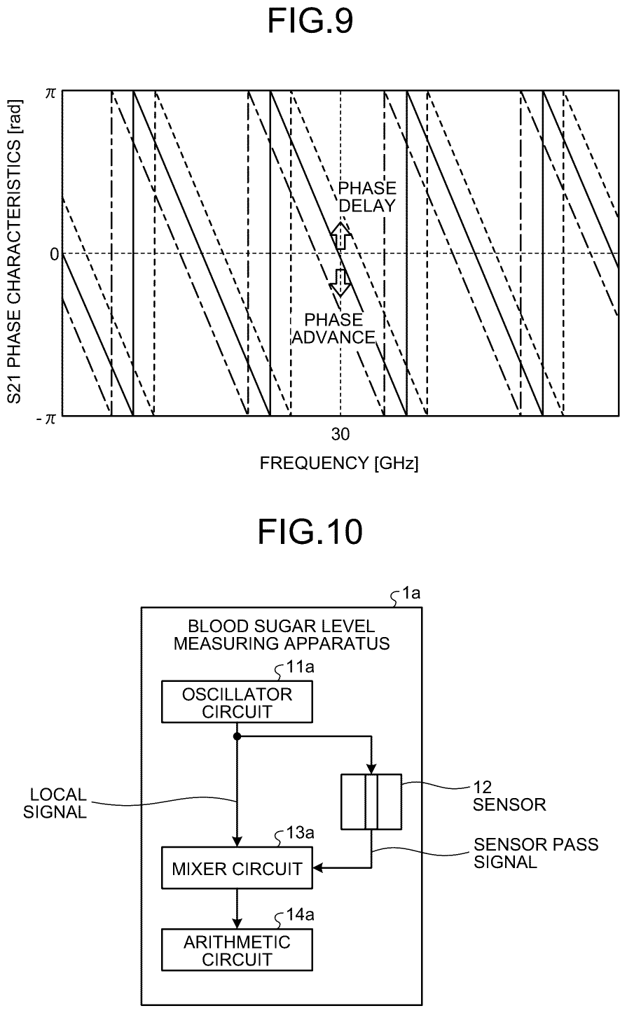

FIG. 9 is a diagram illustrating an example of the relationship between a change in phase and a change in frequency of a sensor pass signal in the first and second embodiments. In this figure, the horizontal axis indicates the frequency and the vertical axis indicates the S21 phase characteristic.

As illustrated in FIG. 9, when the wavelength of the sensor pass signal changes in accordance with the permittivity of the skin, the change can be observed not only as a change in phase but also as a change in frequency. For example, when the wavelength becomes shorter, the phase is advanced and the frequency becomes lower. When the wavelength becomes longer, the phase is delayed and the frequency becomes higher.

A blood sugar level measuring apparatus 1a of the second embodiment observes a change in wavelength of the sensor pass signal as a change in frequency and acquires the measurement value of blood sugar level based on the change in frequency. The blood sugar level measuring apparatus 1a of the second embodiment will be explained below. Points similar or identical to those in the first embodiment will not be further elaborated or be described in a simplified manner.

FIG. 10 is a schematic diagram illustrating an example of the configuration of the blood sugar level measuring apparatus 1a of the second embodiment. As illustrated in this figure, the blood sugar level measuring apparatus 1a includes an oscillator circuit 11a, a sensor 12, a mixer circuit 13a, and an arithmetic circuit 14a.

The oscillator circuit 11a produces an AC signal whose frequency changes with time, that is, a chirp signal. The frequency range of the chirp signal produced by the oscillator circuit 11 is selected from the range in which the permittivity of the skin can change in accordance with blood sugar levels. The oscillator circuit 11a, for example, produces a chirp signal whose frequency changes in a frequency range selected from the range 310 in FIG. 1. The frequency range of the chirp signal produced by the oscillator circuit 11a may be selected from a range other than the range 310.

The chirp signal produced by the oscillator circuit 11a is input as a local signal to the mixer circuit 13a via one of the two split transmission lines. The chirp signal produced by the oscillator circuit 11a is input to the input end of the first signal line 122 of the sensor 12 via the other of the two split transmission lines. The chirp signal output from the output end of the first signal line 122 of the sensor 12 is input as a sensor pass signal to the mixer circuit 13a.

The mixer circuit 13a generates a beat frequency signal indicating the frequency difference between the sensor pass signal and the local signal, and inputs the generated beat frequency signal to the arithmetic circuit 14a.

The arithmetic circuit 14a uses the beat frequency signal, instead of the phase difference Rx used by the arithmetic circuit 14 in the first embodiment, to acquire the measurement value of blood sugar level. The arithmetic circuit 14a can output the acquired measurement value of blood sugar level by any method, in the same manner as the arithmetic circuit 14 in the first embodiment.

In this way, according to the second embodiment, the blood sugar level measuring apparatus 1a includes the mixer circuit 13a that outputs a beat frequency signal indicating the frequency difference between the sensor pass signal and the local signal, and the arithmetic circuit 14a acquires the measurement value of blood sugar level based on the beat frequency signal.

Thus, blood sugar levels can be measured non-invasively in the same manner as in the first embodiment.

Modifications applicable to the first and second embodiments will be described below.

### First Modification

The sensor 12 in the first and second embodiments can be modified in various ways. Instead of the sensor 12 in the first and second embodiments, a sensor 12a of a first modification described below can be applied.

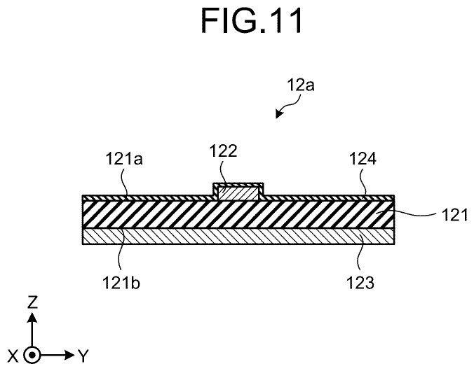

FIG. 11 is a cross-sectional view of the sensor 12a of the first modification cut in the YZ plane. In the sensor 12a, the face 121a and the first signal line 122 are covered with an insulator coating 124. During measurement of blood sugar levels, the subject's skin 200 is pressed against the first signal line 122 through the coating 124.

The coating 124 can be made of any material that has insulating properties. For example, the coating 124 can be made of solder resist. Alternatively, the coating 124 may be made of an insulating ceramic such as silicon oxide.

In this way, according to the first modification, the sensor 12a is configured such that the first signal line 122 is covered with the insulator coating 124 and the subject's skin 200 is pressed against through the coating 124.

Thus, it is possible to prevent damage to the first signal line 122 and corrosion of the first signal line 122 which would be otherwise caused by the subject touching the first signal line 122.

### Second Modification

In the first and second embodiments, the sensor 12 has a structure of a microstrip line. A sensor having a structure of a transmission line other than a microstrip line may be applied to the first and second embodiments. As a second modification, the structures of sensors 12b to 12d that can be applied to the first and second embodiments instead of the sensor 12 will be described.

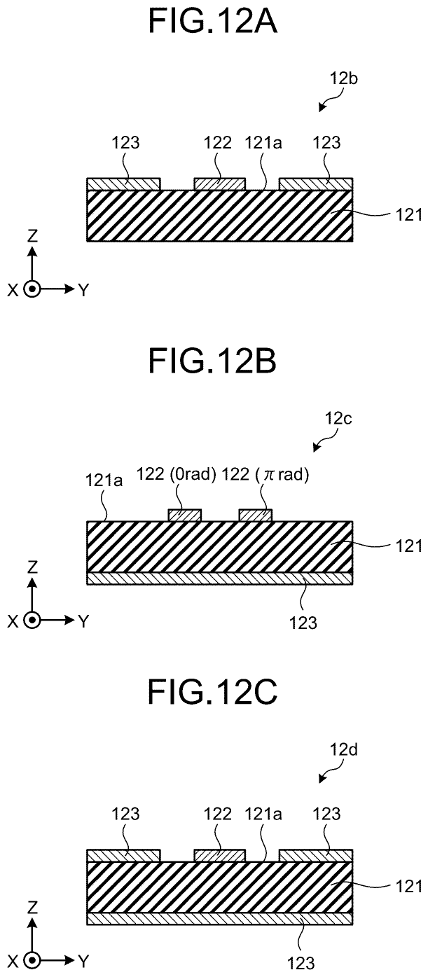

FIGS. 12A to 12C are cross-sectional views of the sensors 12b to 12d of the second modification cut in the YZ plane.

As illustrated in FIG. 12A, the sensor 12b has a structure in which a first signal line 122 and two ground conductors 123 are provided so as to be spaced apart from each other on the face 121a of the first substrate 121. This modification is the same as the first embodiment in that the first signal line 122 is provided on a part of the face 121a of the first substrate 121. In this modification, the ground conductors 123 spaced apart from each other on both sides of the first signal line 122 and having a certain thickness and width are formed to extend in the X direction on a part of the face 121a of the first substrate 121. A transmission line having a structure like the sensor 12b illustrated in this figure is also referred to as a coplanar line.

As illustrated in FIG. 12B, the sensor 12c has a structure in which two first signal lines 122 spaced apart from each other are provided on the face 121a of the first substrate 121, and a ground conductor 123 is provided on the other face 121b of the first substrate 121 and formed over the entire surface of the face 121b. The two first signal lines 122 transmit differential signals, that is, AC signals with inverted phases. A transmission line having a structure like the sensor 12c illustrated in this figure is also referred to as a coplanar stripline.

As illustrated in FIG. 12C, the sensor 12d has a structure in which a first signal line 122 and two ground conductors 123 spaced apart from each other are provided on the face 121a of the first substrate 121. This modification is the same as the first embodiment in that the first signal line 122 is provided on a part of the face 121a of the first substrate 121. In this modification, the ground conductors 123 spaced apart from each other on both sides of the first signal line 122 and having a certain thickness and width are formed to extend in the X direction on a part of the face 121a of the first substrate 121. On the other face 121b of the first substrate 121, a ground conductor 123 is further provided and formed over the entire surface of the face 121b. A transmission line having a structure like the sensor 12d is also referred to as a grounded coplanar line.

In this way, in addition to the microstrip line, transmission line structures such as coplanar line, coplanar stripline, and grounded coplanar line can be applied.

### Third Modification

The shape of the first signal line 122 is not limited to a straight line. As a third modification, the shape of the first signal line 122 that can be applied to the first and second embodiments, other than a straight line shape, will be described.

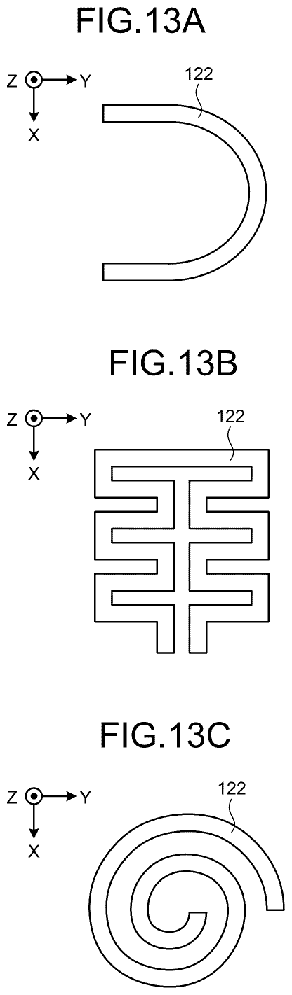

FIGS. 13A to 13C are schematic diagrams illustrating the shape of the first signal line 122 of the third modification. FIGS. 13A to 13C depict the first signal line 122 in various shapes as viewed from the positive side in the Z direction.

The first signal line 122 may have a U-shape as illustrated in FIG. 13A. The first signal line 122 may have a folded shape as illustrated in FIG. 13B. The first signal line 122 may have a spiral shape as illustrated in FIG. 13C.

In this way, the shape of the first signal line 122 can be modified in various ways.

### Fourth Modification

The characteristics of the sensor pass signal can vary depending on the temperature of the sensor 12. Thus, when the subject's skin 200 touches the first signal line 122 of the sensor 12, the temperature of the sensor 12 may change with the subject's body temperature, which may change the results of blood sugar level measurement. As a fourth modification, a sensor 12e that can cancel the effect of temperature change in the sensor 12 caused by the subject's touch will be described. The sensor 12e of the fourth modification can be applied to the first and second embodiments.

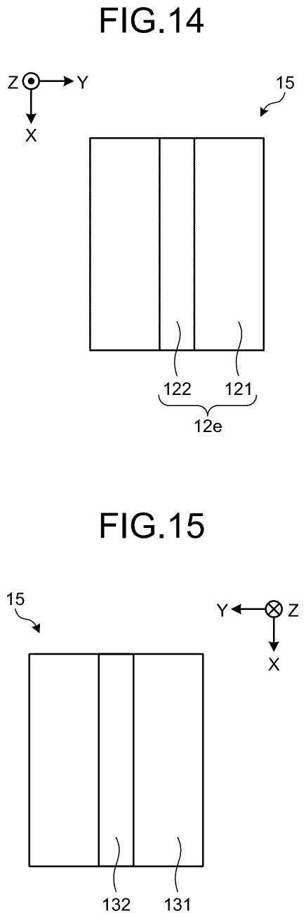

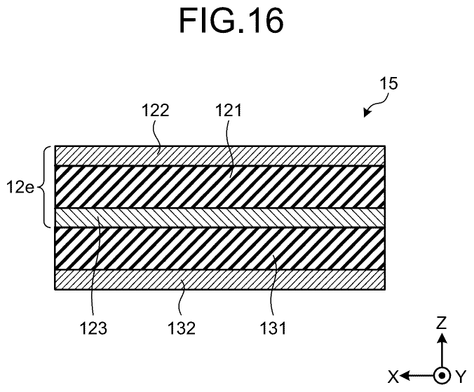

FIGS. 14, 15, and 16 are schematic diagrams illustrating examples of the configuration of the sensor 12e of the fourth modification. According to the fourth modification, the sensor 12e is incorporated in a sensor unit 15. FIG. 14 is a diagram of the sensor unit 15 as viewed from the positive side in the Z direction. FIG. 15 is a diagram of the sensor unit 15 as viewed from the negative side in the Z direction. FIG. 16 is a cross-sectional view of the sensor unit 15 cut in the XZ plane.

The sensor unit 15 includes the sensor 12e. The sensor 12e has the same structure as the sensor 12. That is, on a part of the face 121a of the first substrate 121, a first signal line 122 made of a conductor and extending in the X direction with a certain thickness and width is provided to pass through approximately the center when the first substrate 121 is viewed in a plan view, and on the face 121b of the first substrate 121, a ground conductor 123 is provided and formed over the entire surface of the face 121b.

A second substrate 131 is provided on the negative side in the Z direction of the sensor 12e. In other words, the second substrate 131 is provided so as to be opposed to the face 121b of the first substrate 121 with the ground conductor 123 interposed. The shape of the second substrate 131 and the material forming the second substrate 131 are the same as those of the first substrate 121. A second signal line 132 is provided on a face of the second substrate 131 opposite the ground conductor 123. The second signal line 132 is provided on the back side of the sensor unit 15 as viewed from the first signal line 121. Thus, with the sensor unit 15, the subject is unable to touch the second signal line 132 during measurement of blood sugar levels, but the subject's body temperature can propagate to the first substrate 121, the ground conductor 123, the second substrate 131, and the second signal line 132. The shape of the second signal line 132 and the material forming the second signal line 132 are the same as those of the first signal line 122. That is, the ground conductor 123, the second substrate 131, and the second signal line 132 have a structure of a microstrip line, in the same manner as in the sensor 12e.

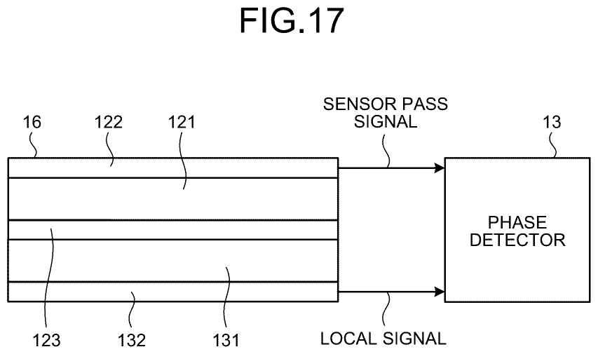

FIG. 17 is a diagram illustrating transmission paths of a sensor pass signal and a local signal when the fourth modification is applied to the first embodiment.

As illustrated in FIG. 17, the AC signal passing through the first signal line 122 is input as a sensor pass signal to the phase detector 13. Further, the AC signal passing through the second signal line 132 is input as a local signal to the phase detector 13.

The phase detector 13 inputs the phase difference Rx between the sensor pass signal and the local signal to the arithmetic circuit 14, as described in the first embodiment. The arithmetic circuit 14 calculates the measurement value of blood sugar level based on the phase difference Rx through the operation described in the first embodiment.

During measurement of blood sugar levels, the subject's skin 200 is pressed against the first signal line 122. The subject's heat then propagates through the entire sensor unit 15, and the temperature becomes almost uniform in the entire sensor unit 15. Thus, the temperature condition can be made equal between the sensor pass signal and the local signal. Since the comparison result between the sensor pass signal and the local signal that pass through the respective transmission lines at the same temperature is used to calculate the measurement value of blood sugar level, the effect of the subject's body temperature on the sensor pass signal can be canceled. In other words, according to the fourth modification, accurate blood sugar level measurement is implemented while suppressing the effect of the subject's body temperature on the sensor 12e.

In FIG. 17, the sensor 12e of the fourth modification is applied to the first embodiment. The sensor 12e of the fourth modification can also be applied to the second embodiment. When the sensor 12e of the fourth modification is applied to the second embodiment, the AC signal passing through the first signal line 122 is input as a sensor pass signal to the mixer circuit 13a, and the AC signal passing through the second signal line 132 is input as a local signal to the mixer circuit 13a, in the same manner as in the example illustrated in FIG. 17.

In the first and second embodiments and the modifications thereof, the measuring apparatus that measures blood sugar levels as biological information has been described. Biological information other than blood sugar levels may be measured.

For example, the permittivity of the skin may be used as biological information to be measured. The measuring apparatus of embodiments may acquire the permittivity of the skin based on the phase difference or frequency difference between the sensor pass signal and the local signal, and output the acquired permittivity of the skin.

The permittivity of the skin can also be affected by the amount of cancer cells. Thus, the measuring apparatus of embodiments may be configured to measure the amount of cancer cells as biological information. The measuring apparatus of embodiments may acquire the amount of cancer cells based on the phase difference or frequency difference between the sensor pass signal and the local signal, and output the acquired amount of cancer cells.

As described in the first and second embodiments and the modifications thereof, the measuring apparatus includes a first substrate (for example, first substrate 121) made of a dielectric and having a ground conductor (for example, ground conductor 123) and a first signal line (for example, first signal line 122) against which a living body is to be pressed, an oscillator circuit (for example, oscillator circuit 11, 11a) configured to produce an AC signal, and an arithmetic circuit (for example, arithmetic circuit 14, 14a) configured to acquire biological information based on a comparison between the AC signal that passes through the first signal line and the AC signal that does not pass through the first signal line. The comparison between the AC signal that passes through the first signal line and the AC signal that does not pass through the first signal line is to detect the phase difference Rx in the first embodiment and to detect the frequency difference in the second embodiment.

Thus, biological information can be measured non-invasively.

An embodiment provides an advantageous effect that it is possible to provide a measuring apparatus and a measurement method that can measure biological information non-invasively.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A measuring apparatus comprising:
a first substrate made of a dielectric and having a first signal line against which a living body is to be pressed, and a ground conductor;
an oscillator circuit that produces a first signal of alternating current; and
an arithmetic circuit that acquires biological information based on a comparison between a second signal and a third signal, the second signal corresponding to the first signal that has passed through the first signal line, the third signal corresponding to the first signal that did not pass through the first signal line.

2. The measuring apparatus according to claim 1, wherein the first signal line and the ground conductor are provided so as to be spaced apart from each other on the first substrate, and a surface area of the ground conductor is larger than a surface area of the first signal line when viewed in a plan view.

3. The measuring apparatus according to claim 1, wherein the first signal line is covered with an insulator coating, and a living body is pressed against the first signal line through the coating.

4. The measuring apparatus according to claim 1, further comprising a phase detector that detects a phase difference between the second signal and the third signal, wherein
the arithmetic circuit acquires the biological information based on the phase difference.

5. The measuring apparatus according to claim 1, wherein
the first signal is a chirp signal,
the measuring apparatus further comprises a mixer circuit into which the second signal and the third signal are input, and that outputs a signal corresponding to a frequency difference between the second signal and the third signal, and
the arithmetic circuit acquires the biological information based on the signal corresponding to the frequency difference.

6. The measuring apparatus according to claim 1, wherein the first signal line is provided on a first face of the first substrate, and the ground conductor is provided on a second face of the first substrate opposite to the first face.

7. The measuring apparatus according to claim 6, further comprising:
a second substrate provided at a position opposed to the second face, the ground conductor being sandwiched between the first substrate and the second substrate; and
a second signal line against which a living body is not to be pressed, provided on a face of the second substrate opposite to a face on which the ground conductor is provided,
wherein the third signal corresponds to the first signal that passes through the second signal line.

8. The measuring apparatus according to claim 7, wherein the biological information is a permittivity of a living body, a blood sugar level of a living body, or an amount of cancer cells in a living body.

9. A measuring method comprising:
generating a first signal of alternating current;
causing a living body to be pressed against a first signal line on a first substrate made of a dielectric, the first substrate further having a ground conductor; and
acquiring biological information of the living body based on a comparison between a second signal and a third signal in a state in which the living body is pressed against the first signal line, the second signal corresponding to the first signal of alternating current that has passed through the first signal line, the third signal corresponding to the first signal that did not pass through the first signal line.
